(19) United States
(12) Patent Application Publication
(10) Pub. No.: US 2026/0060699 A1
(43) Pub. Date: Mar. 5, 2026

(54) THROMBUS REMOVAL SYSTEMS AND ASSOCIATED METHODS

(71) Applicant: Shifamed Holdings, LLC, Campbell, CA (US)

(72) Inventor: Paul GUNNING, Montara, CA (US)

(21) Appl. No.: 19/106,179

(22) PCT Filed: Aug. 18, 2023
(86) PCT No.: PCT/US2023/072484

§ 371 Date: Feb. 24, 2025

Related U.S. Application Data
(60) Provisional application No. 63/373,415, filed on Aug. 24, 2022.

(51) Int. Cl. A61B 17/22; A61B 17/00; A61B 17/3203

(52) U.S. Cl. CPC A61B 17/22; A61B 17/32037; A61B 2017/00557; A61B 2017/00561; A61B 2017/00778; A61B 2017/00867; A61B 2017/22051; A61B 2017/22079; A61B 2217/005

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### PRIORITY CLAIM

This patent application claims priority to U.S. provisional patent application No. 63/373,415, titled “THROMBUS REMOVAL SYSTEMS AND ASSOCIATED METHODS”, and filed on Aug. 24, 2022, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the tortuous vascular anatomy, may not be effective in removing thrombotic material, and/or may lack the ability to provide sensor data or other feedback to the clinician during the thrombectomy procedure. Existing thrombectomy devices operate based on simple aspiration which works sufficiently for certain clots but is largely ineffective for difficult, organized clots. Many patients presenting with deep vein thrombus (DVT) are left untreated as long as the risk of limb ischemia is low. In more urgent cases, they are treated with catheter-directed thrombolysis or lytic therapy to break up a clot over the course of many hours or days. More recently other tools like clot retrievers have been developed to treat DVT and pulmonary embolism (PE), but these tools are not being widely adopted because of their limited effectiveness and additional costs versus aspiration or the standard of case. Other recent developments focus on slicing or macerating the clot, but these mechanisms are designed to reduce the risk of the catheter clogging and do not address the problem of tough, large, organized clots. There remains the need for a device to address these and other problems with existing venous thrombectomy including, but not limited to, a fast, easy-to-use, and effective device for removing a variety of clot morphologies.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports and fluid streams of a thrombus removal system according to embodiments of the present technology.

FIGS. 4A-4C illustrate various embodiments of a thrombus removal system including a saline source, an aspiration system, and one or more controls for controlling irrigation and/or aspiration of the system.

FIG. 5 is one embodiment of a funnel of a thrombus removal system that includes directional features configured to cause twisting, tumbling, or apply rotational forces to a clot within the funnel during aspiration.

FIGS. 6A-6B illustrate embodiments of a funnel with directional features and one or more jets.

FIGS. 7A-7B and 8A-8B illustrate additional embodiments of a funnel with directional features and one or more jets.

### SUMMARY OF THE DISCLOSURE

A thrombus removal is provided, comprising an elongate shaft comprising a working end, at least one fluid lumen in the elongate shaft, and two or more apertures disposed at or near the working end, the two or more apertures in fluid communication with the least one fluid lumen and configured to generate two or more fluid streams to mechanically fractionate a target thrombus.

A thrombus removal device is provided, comprising: an elongate shaft comprising a working end; at least one aspiration lumen in the elongate shaft; a funnel disposed at or near the working end, the funnel comprising directional features configured to impart directional motion upon a clot or direct flow within the funnel while aspiration is activated; and one or more jet ports disposed in the funnel, the jet ports being fluidly coupled to a fluid source, the jet ports being configured to direct one or more fluid streams into the funnel.

In some aspects, the directional features comprise ribs extending from an interior surface of the funnel.

In another aspect, the directional features are arranged in a spiral or helical pattern.

In some aspects, the directional features are continuous from a distal end of the funnel until the aspiration lumen.

In one aspect, the directional features are embedded within a compliant material of the funnel.

In some aspects, the directional features are configured to provide structural support for the funnel.

In one aspect, the directional features comprise a shape memory material that is configured to self-expand the funnel during deployment of the device.

In some aspects, the directional features comprise inflatable members.

In some aspects, the inflatable members are fluidly coupled to the fluid source, wherein the fluid source is configured to deliver fluid to the inflatable members to inflate the inflatable members.

In one aspect, the one or more jet ports are disposed on the inflatable members.

In some aspects, the inflatable members are fluidly coupled to the fluid source, wherein the fluid source is configured to deliver fluid to the inflatable members to inflate the inflatable members and to direct the one or more fluid streams into the funnel.

In other aspects, the jets ports are configured to generate fluid streams in the same direction as the directional motion imparted on clots by the directional features.

In one aspect, the jets ports are configured to generate fluid streams in the opposite direction as the directional motion imparted on clots by the directional features.

In some aspects, the jet ports are configured to generate fluid streams that cross the funnel.

In one aspect, the jet ports are offset so as to cause shearing of the clot.

A method for removing a thrombus from a blood vessel of a patient with a thrombus removal device is provided, the method comprising introducing a funnel of a thrombus removal device to a thrombus location in a blood vessel; operating an aspiration source of the thrombus removal device to at least partially capture a thrombus in the funnel; imparting a tumbling or twisting motion to the thrombus with one or more directional features of the funnel; directing one or more fluid streams into the funnel; and aspirating at least a portion of the thrombus into the thrombus removal device.

In some aspects, the fluid streams are directed in the same direction as the tumbling or twisting motion of the thrombus.

In another aspect, the fluid streams are applied in the opposite direction as the tumbling or twisting motion of the thrombus.

In some aspects, the method includes inflating the one or more directional features with a fluid.

In one aspect, the one or more fluid streams originate from jet ports disposed on the directional features.

In another aspect, the method includes delivering fluid from a fluid source to the funnel to inflate the one or more directional features and direct the one or more fluid streams.

### DETAILED DESCRIPTION

This application is related to disclosure in International Application No. PCT/US2021/020915, filed Mar. 4, 2021 (the '915 application), and International Application No. PCT/US2022/033024, filed Jun. 10, 2022 (the '024 application), the disclosures of which are incorporated by reference herein for all purposes. The '915 and '024 applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section.

Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance.

Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body.

The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

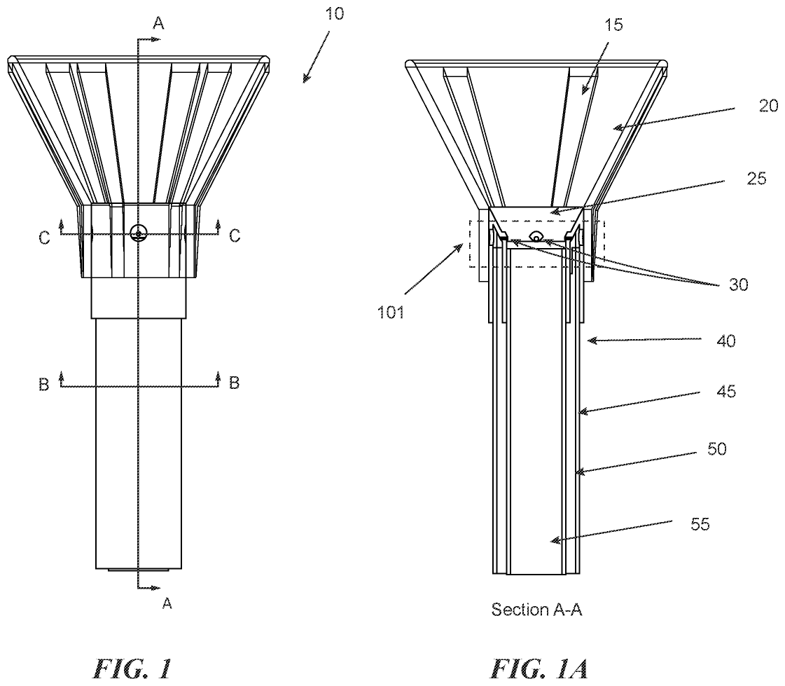

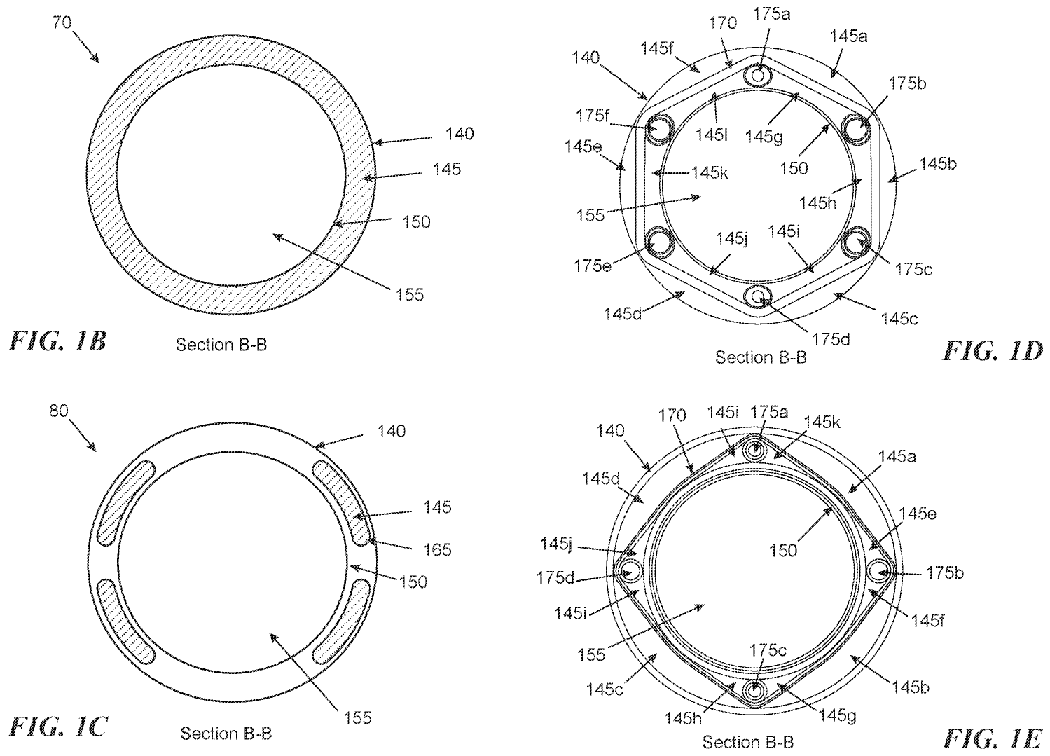

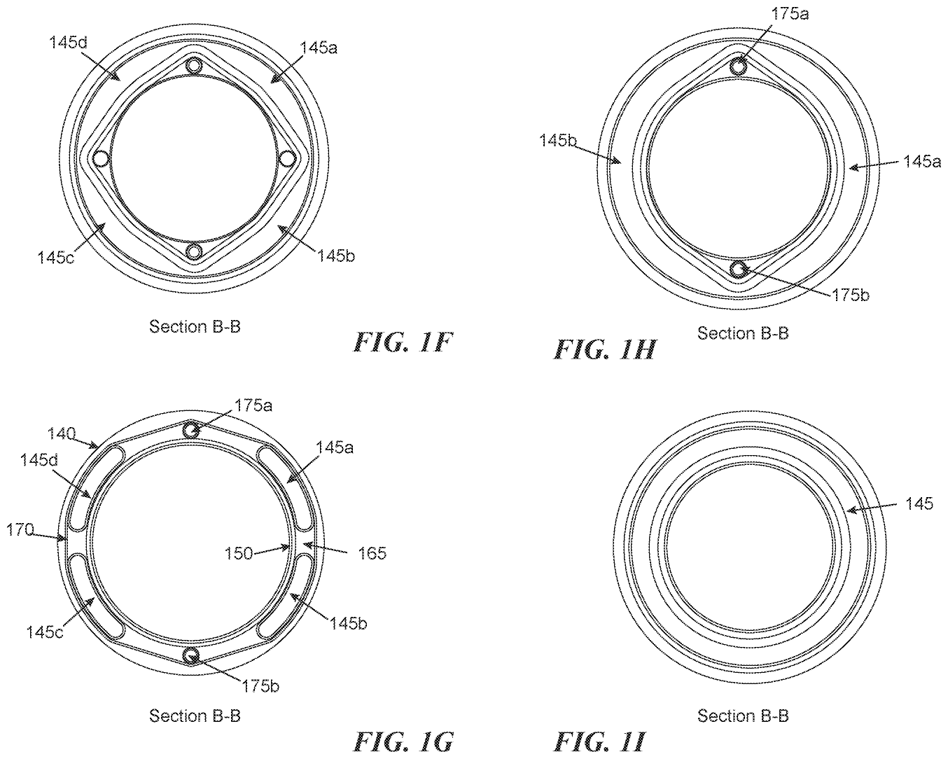

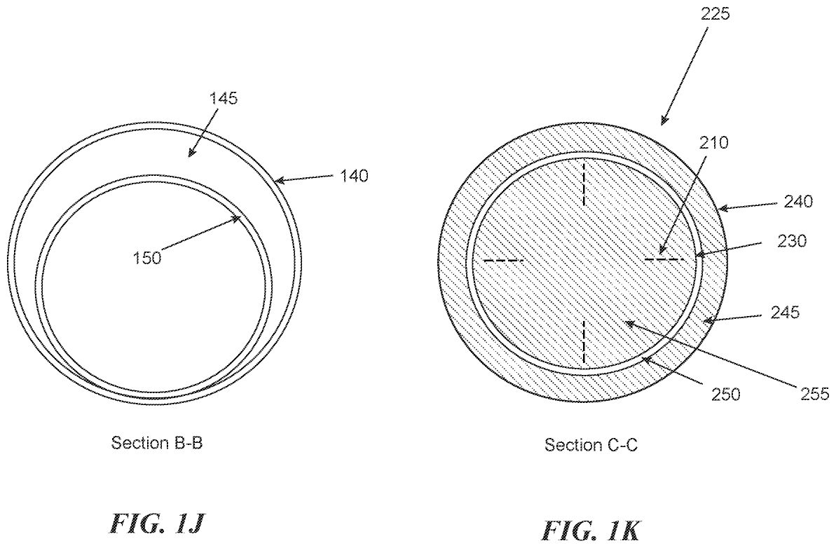

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The funnel can have a variety of shapes and constructions as would be understood by one of skill from the description herein. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures are no smaller than 0.0100″ or even as small as 0.008″ to avoid undesirable spraying of fluid. In some embodiments, the system can have a minimum vacuum or aspiration pressure of 15 inHg, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally similar features and components described herein may be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plain view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-14l and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injections to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

In various embodiments, the fluid pressure is generated at the pump (in the console or handle). The fluid is accelerated as it exits the ports at the distal end and is directed to the target clot. In this way a wider variety of cost-effective components can be used to form the catheter while still maintaining a highly-effective device for clot removal. Additional details are provided below.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the exemplary outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plain view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

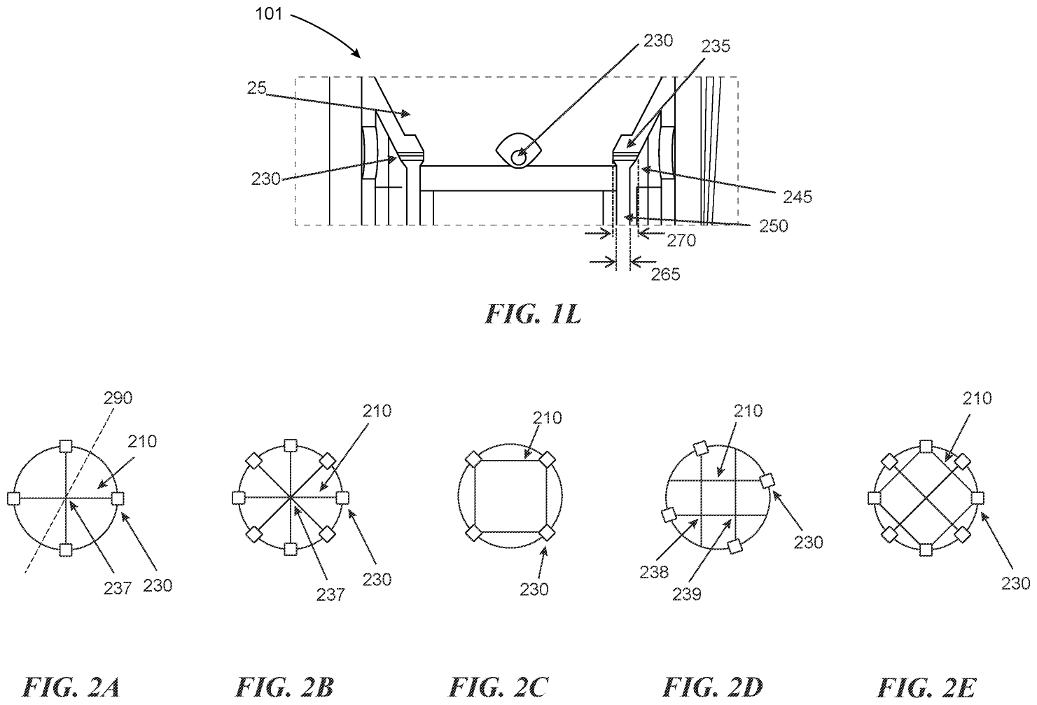

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which multiply with respect to individual fluid streams that are not directed to combine at the intersection. The increased fluid momentum and/or energy transfer at an intersection may advantageously fragment thrombus more efficiently and/or quickly. As described above, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g. above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e. speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

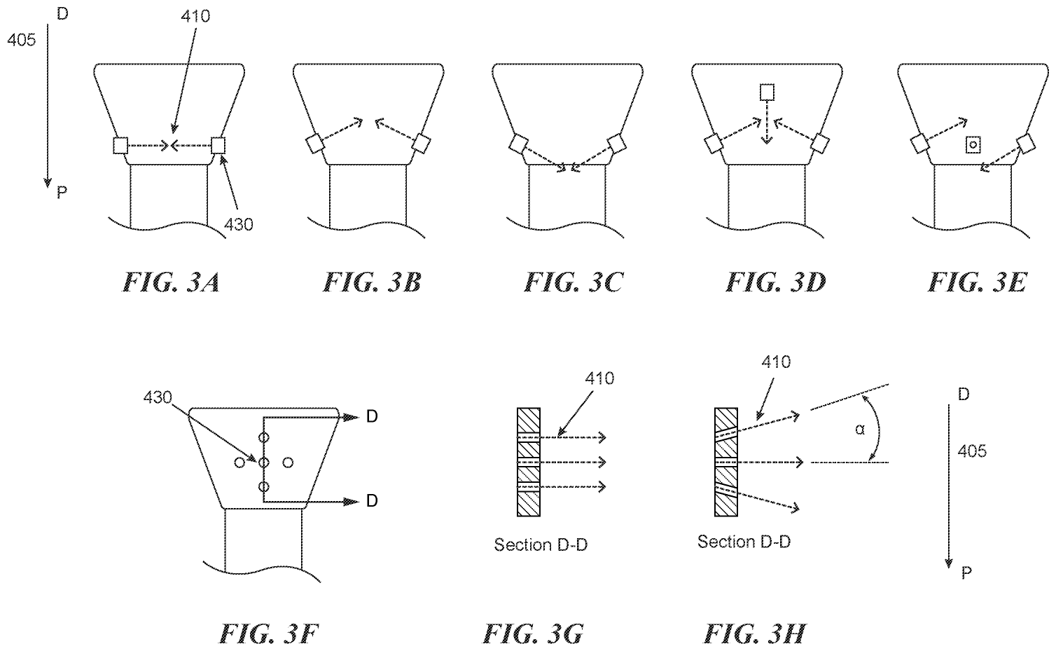

FIGS. 3A-3H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle α may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

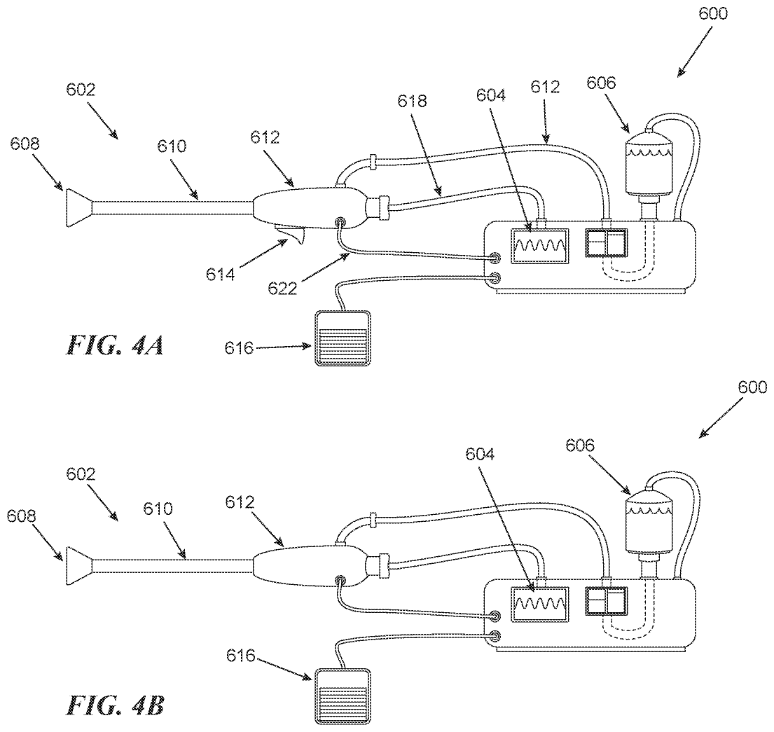

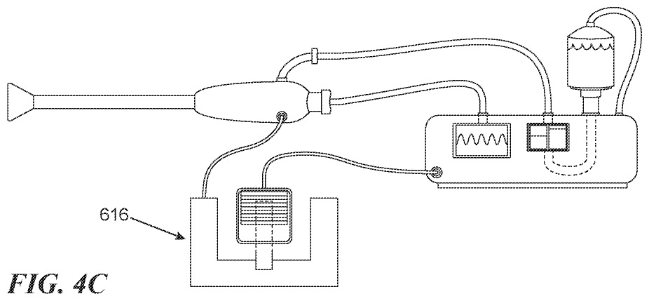

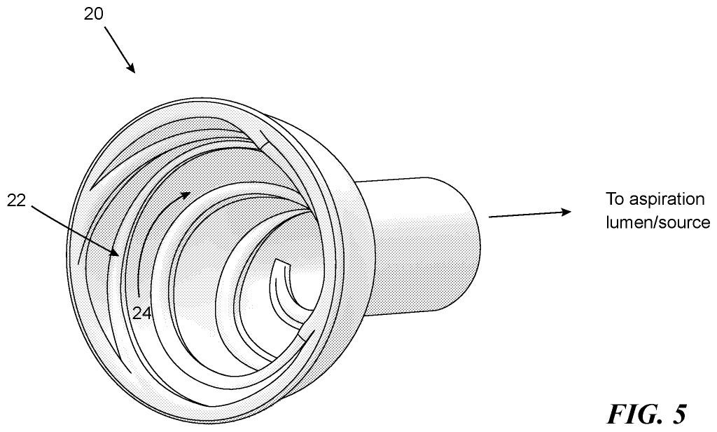

FIGS. 4A-4C illustrate various configurations of a thrombus removal system 600, including a thrombus removal device, 602, a vacuum source and cannister 604, and a fluid source 606. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 4A, the device can include a finger switch or trigger 614 and a foot pedal or switch 616. These can be used to control aspiration and irrigation, respectively. Alternatively, as shown in the embodiment of FIG. 4B, the device can include only a foot switch 614, which can be used to control both functions, or in FIG. 4C, the device can include only an overpedal 616, also used to control both functions. It is also contemplated that an embodiment could include only a finger switch to control both aspiration and irrigation functions. As shown in FIG. 4A, the vacuum source can be coupled to the aspiration lumen of the device with a vacuum line 618. Any clots or other debris removed from a patient during therapy can be stored in the vacuum cannister 604. Similarly, the fluid source (e.g., a saline bag) can be coupled to the fluid lumens of the device with a fluid line 620.

Still referring to FIG. 4A, electronics line 622 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both systems in response to sensor data, such as pressure data, flow data, etc.

As is described above, aspiration occurs down the central lumen of the device and is provided by a vacuum pump in the console. The vacuum pump can include a container that collects any thrombus or debris removed from the patient.

### Funnel With Directional Features

FIG. 5 shows one embodiment of a funnel 20 of a thrombus removal device that includes directional features 22 configured to cause twisting or tumbling of a clot that enters the funnel during aspiration. In some embodiments, the directional features apply rotational force or torque to the clot via direct interaction with the clot to cause it to spiral, spin, or swirl along the interior surface of the clot as it approaches the aspiration lumen of the device.

In the illustrated embodiment, the directional features 22 can comprise ribs, protrusions, or rails that extend outwards into the tunnel from the interior surface of the funnel. As described previously, the funnel can generally comprise a frame structure (not shown) and a compliant material disposed over or around the frame structure. The frame structure is generally designed and configured to provide structural support for the funnel, and also can be configured to assist or perform expansion of the funnel when the thrombus removal device is deployed at a target thrombus location. For example, the frame structure can comprise a shape memory material such as nitinol. The compliant material can comprise a urethane such as Chronoflex, for example. In some embodiments, the directional features can also be embedded in or surrounded by the compliant material. In other embodiments, the directional features and the frame structure can be the same physical structure, i.e., the frame structure can provide structural support for the funnel and automatically expand the funnel, and can also be shaped and configured to extend from the inner surface of the funnel to cause a thrombus to tumble, twist, or spin within the funnel during aspiration.

In some embodiments, the directional features 22 can comprise inflatable members. The inflatable members can be configured to be inflated or expanded from a collapsed or unexpanded state to an expanded or inflated state. Inflation can be controlled, for example, by injecting a gas or fluid (e.g., saline), into the inflatable members. The gas or fluid can be directed into the funnel/inflatable members with the fluid lumens or other lumens in the device described herein. Inflation of the directional features can provide structural support or rigidity to the funnel, in addition to providing the expanded directional features that apply torque or rotational force to a clot within the funnel. In any of the embodiments described herein, fluid jets or ports can be positioned within the inflatable members or within the funnel. In either embodiment, directing saline or a fluid into the funnel and/or inflatable members can serve the dual purpose of both inflating the inflatable members/funnel and also delivering jets or fluid streams out of the ports in the funnel/inflatable members to interact with, macerate, cut up, or impart motion into the clot.

Generally, the directional features are configured to protrude or stand proud from an interior surface of the funnel. However, in other embodiments, the directional features can be recessed into the funnel, in the form of grooves, channels, or craters. In the embodiment of FIG. 5, the directional features comprise a generally spiral or helical shape that winds in a generally clockwise direction in the funnel from the distal end of the funnel to the proximal end of the funnel and aspiration lumen (when viewing the funnel from the top-down view as shown). However, it should be understood that these directional features can also be configured to wind or turn in a counter-clockwise direction in the same view. While the embodiment of FIG. 5 illustrates a smooth and continuous helical or spiral shape, it should be understood that in other embodiments the directional features need not be continuous throughout the funnel. For example, the directional features could comprise bumps, dots, helical segments, or the like.

Generally, the purpose of the directional features is to impart, by physical contact or interference with the clot, some sort of tumbling, spinning, or disruption to the clot to cause chaotic or unpredictable movement of the clot within the funnel during aspiration. Referring still to FIG. 5, the helical winding nature of the illustrated directional features can generally impart movement along a path indicated by arrow 24. The force applied by the directional features 22 to the clot or clots within the funnel can assist in breaking up the clots within the funnel, particularly when the clot engages with jets of the thrombus device (to be described below). Ultimately, the end goal of the device, and the funnel with directional features, is to encourage or facilitate the clot along a path that ends at the aspiration lumen and/or jets of the thrombus removal device. The directional features can prevent the clot, particularly large clots, from getting stuck or lodged in the funnel prior to reaching the jets or aspiration lumen.

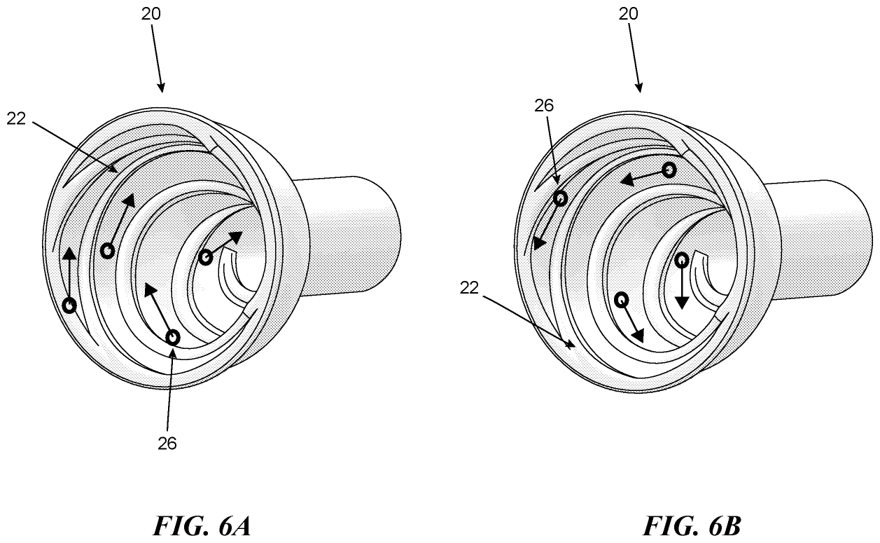

FIGS. 6A-6B illustrate additional embodiments of a funnel with directional features 22, similar to as described above. Generally, the directional features 22 of FIGS. 6A-6B function to cause one or more clots to tumble or twist within the funnel generally along a tortuous path in the clockwise direction (when looking top down at the distal end of the funnel). In the embodiments of FIGS. 6A-6B, the funnel can include one or more jet ports 26 fluidly coupled to a fluid source of the thrombus removal device via one or more fluid lumens in the device shaft. These jet ports can be arranged and configured to deliver fluid streams into the funnel, indicated by the arrows. In some embodiments, the fluid delivered to the jet ports can serve to both provide fluid streams into the funnel and also inflate the directional features 22 of the funnel.

Generally, in embodiments herein that include jets, the fluid pressure of the jets can be controlled depending on the intended use or function of the jets. For example, jets located proximally within the funnel towards or at the aspiration lumen may have high fluid pressures since it may be desirable to break up, macerate, or destroy clots near the aspiration lumen to remove the clot. However, jets located within the funnel or more distally towards the end of the funnel may be produced with lower fluid pressures. These jets with lower pressures may be intended to support imparting spin or movement on the clot and/or providing irrigation to the funnel and clot. In some aspects, devices and systems herein can include any combination of high and/or low pressure jets to assist with clot removal.

In the embodiment of FIG. 6A, the jet ports 26 of the funnel are generally arranged to produce fluid streams in the same direction that the directional features are intended to direct the clot. Specifically, the directional features are configured to encourage the clots along a winding helical path (represented by arrow 24 in FIG. 5), and the jets in FIG. 6A are designed and configured to push the clots along in the same path as the directional features (e.g., in a clockwise direction when viewing the funnel in the top down view as shown).

In contrast, the jet ports of FIG. 6B are pointed in the opposite direction of the jet ports of FIG. 6A. Instead of assisting or enhancing the directional features like in the FIG. 6A embodiment, the jet ports and fluid streams of the FIG. 6B embodiment direct fluid in the opposite direction. So while the directional features of the FIG. 6B embodiment encourage the clot in a clockwise path around the interior surface of the clot (when viewing top down from the distal end), the jet ports 26 point in the opposite direction and violently collide with any clots tumbling or twisting towards the aspiration lumen. This interaction between the direction of the clot cause by the directional features and the opposing fluid streams from jet ports 26 can assist and aid in breaking up or macerating the clots during aspiration.

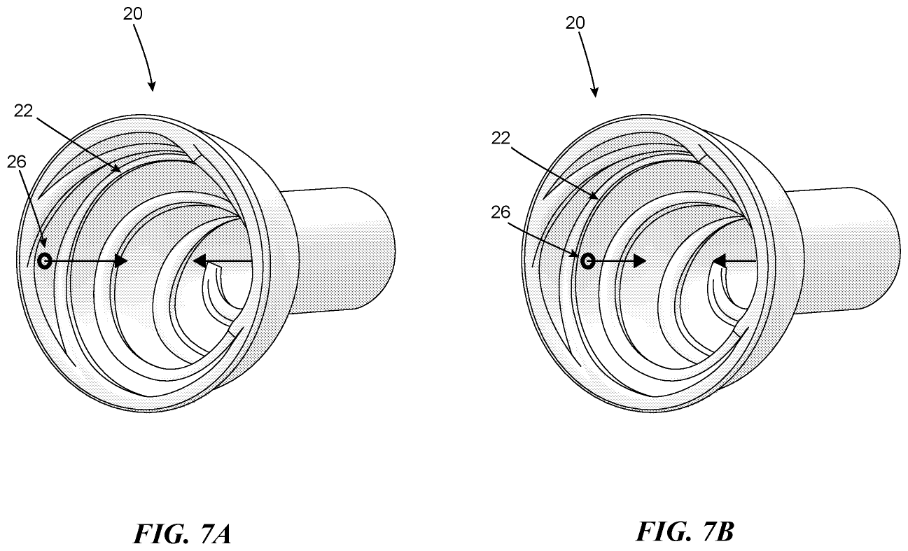

FIGS. 7A-7B illustrate another embodiment of a funnel 20 with directional features 22 and jet ports 26. In this embodiment, the jets don't directly assist or oppose tumbling or twisting of the clots, but instead are directed across the interior of the funnel. The embodiment of FIG. 7A shows the jet ports near a distal end of the funnel and directed across the opening of the funnel at each other, and the embodiment of FIG. 7B shows the jet ports positioned more proximally in the funnel closer to the aspiration lumen. In other embodiments, not shown, the same configuration of jets can be at or even within the aspiration lumen itself. It should also be understood that while these jets are directed at each other, in other embodiments the jets can be offset from one another so as to not cause colliding jet streams, but instead cause jet streams that pass each other before reaching the opposite side of the funnel or colliding with a clot. In these offset jet embodiments, the passing jets can create a shearing motion or energy that can further assist with tumbling the jets and macerating or cutting up the clots during aspiration. In some embodiments, the fluid delivered to the jet ports can serve to both provide fluid streams into the funnel and also inflate the directional features 22 of the funnel and provide irrigant to clear the aspiration lumen.

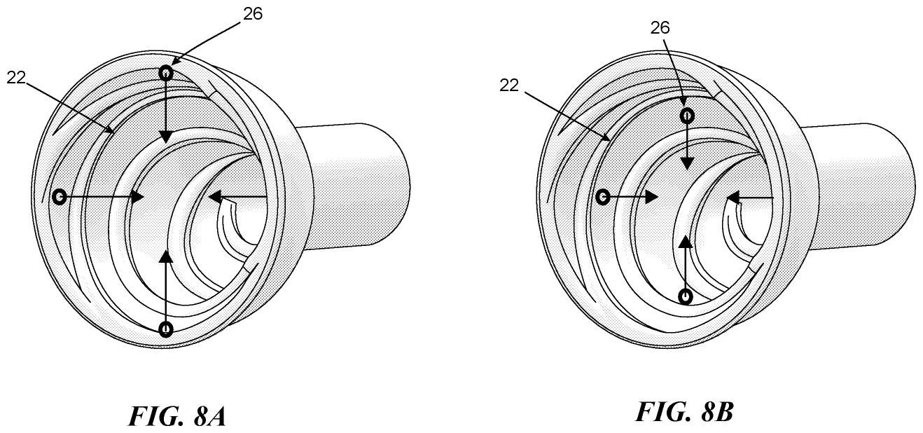

FIGS. 8A-8B illustrate another embodiment of a funnel 20 with directional features 22 and jet ports 26. This embodiment is similar to the one illustrated and described above with respect to FIGS. 7A-7B. However, this embodiment includes at least four intersecting and/or colliding jet ports and fluid streams. Similar to the embodiment of FIGS. 7A-7B, the fluid streams of this embodiment can be configured to intersect and collide, or they can be offset or pointed in a direction so that the fluid streams do not collide or intersect. Generally, any of the fluid stream configurations described herein, such as in FIGS. 2A-2E or 3A-3H can be implemented in the funnel or aspiration lumen along with the directional features described above. In some embodiments, the fluid delivered to the jet ports can serve to both provide fluid streams into the funnel and also inflate the directional features 22 of the funnel.

While the embodiments herein have been described as being intended to remove thrombi from a patient's vasculature, other applications of this technology are provided. For example, the devices described herein can be used for breaking up and removing hardened stool from the digestive tract of a patient, such as from the intestines or colon of a patient. In one embodiment, the device can be inserted into a colon or intestine of the patient (such as through the anus) and advanced to the site of hardened stool. Next, the aspiration system can be activated to engage the hardened stool with an engagement member (e.g., funnel) of the device. Finally, the jets or irrigation can be activated to break off pieces of the hardened stool and aspirate them into the system. Any of the techniques described above with respect to controlling the system or removing clots can be applied to the removal of hardened stool.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

### Conclusion

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or”as in “A and/or B”refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A thrombus removal device, comprising:
an elongate shaft comprising a working end;
at least one aspiration lumen in the elongate shaft;
a funnel disposed at or near the working end, the funnel comprising directional features configured to impart directional motion upon a clot or direct flow within the funnel while aspiration is activated; and
one or more jet ports disposed in the funnel, the jet ports being fluidly coupled to a fluid source, the jet ports being configured to direct one or more fluid streams into the funnel.

2. The device of claim 1, wherein the directional features comprise ribs extending from an interior surface of the funnel.

3. The device of claim 1, wherein the directional features are arranged in a spiral or helical pattern.

4. The device of claim 3, wherein the directional features are continuous from a distal end of the funnel until the aspiration lumen.

5. The device of claim 1, wherein the directional features are embedded within a compliant material of the funnel.

6. The device of claim 1, wherein the directional features are configured to provide structural support for the funnel.

7. The device of claim 1, wherein the directional features comprise a shape memory material that is configured to self-expand the funnel during deployment of the device.

8. The device of claim 1, wherein the directional features comprise inflatable members.

9. The device of claim 8, wherein the inflatable members are fluidly coupled to the fluid source, wherein the fluid source is configured to deliver fluid to the inflatable members to inflate the inflatable members.

10. The device of claim 8, wherein the one or more jet ports are disposed on the inflatable members.

11. The device of claim 8, wherein the inflatable members are fluidly coupled to the fluid source, wherein the fluid source is configured to deliver fluid to the inflatable members to inflate the inflatable members and to direct the one or more fluid streams into the funnel.

12. The device of claim 1, wherein the jets ports are configured to generate fluid streams in the same direction as the directional motion imparted on clots by the directional features.

13. The device of claim 1, wherein the jets ports are configured to generate fluid streams in the opposite direction as the directional motion imparted on clots by the directional features.

14. The device of claim 1, wherein the jet ports are configured to generate fluid streams that cross the funnel.

15. The device of claim 1, wherein the jet ports are offset so as to cause shearing of the clot.

16. A method for removing a thrombus from a blood vessel of a patient with a thrombus removal device, the method comprising:
introducing a funnel of a thrombus removal device to a thrombus location in a blood vessel;
operating an aspiration source of the thrombus removal device to at least partially capture a thrombus in the funnel;
imparting a tumbling or twisting motion to the thrombus with one or more directional features of the funnel;
directing one or more fluid streams into the funnel; and
aspirating at least a portion of the thrombus into the thrombus removal device.

17. The method of claim 16, wherein the fluid streams are directed in the same direction as the tumbling or twisting motion of the thrombus.

18. The method of claim 16, wherein the fluid streams are applied in the opposite direction as the tumbling or twisting motion of the thrombus.

19. The method of claim 16, further comprising inflating the one or more directional features with a fluid.

20. The method of claim 19, wherein the one or more fluid streams originate from jet ports disposed on the directional features.

21. The method of claim 20, further comprising delivering fluid from a fluid source to the funnel to inflate the one or more directional features and direct the one or more fluid streams.
